(19) United States
(12) Patent Application Publication
(10) Pub. No.: US 2026/0263967 A1
(43) Pub. Date: Sep. 10, 2026

(54) APPARATUS FOR DETERMINING WATER SEPARATION CHARACTERISTICS OF HYDROCARBON FUELS AND RELATED METHOD

(71) Applicant: EMCEE ELECTRONICS, INC., Venice, FL (US)

(72) Inventor: Ryan Matthew Case, Sarasota, FL (US)

(21) Appl. No.: 19/562,015

(22) Filed: Mar. 10, 2026

Related U.S. Application Data
(60) Provisional application No. 63/769,224, filed on Mar. 10, 2025.

(51) Int. Cl. B01D 17/04; G01N 15/00; G01N 33/22; G01N 33/28

(52) U.S. Cl. CPC B01D 17/045; G01N 15/00; G01N 33/22; G01N 33/28; G01N 2015/003

(57) ABSTRACT

A coalescer element for determining water separation characteristics of hydrocarbon fluids includes a first body portion with a fluid conduit having an inlet and outlet, and a second body portion with a corresponding fluid conduit. A filter separator pad is disposed between the outlet of the second body portion and the inlet of the first body portion. The first body portion includes a cylindrical wall with an open top and closed bottom, receiving the second body portion in a fluid-tight relationship secured by an O-ring seal. The filter separator pad includes stacked fiberglass discs with scrim reinforcement. In operation, a water/fuel emulsion passes through the aligned fluid conduits and coalescer discs, enabling measurement of water separability as an indicator of surfactant presence in the fuel. The apparatus provides simplified testing methods while reducing operator skill requirements for evaluating water separation effectiveness in transient fuel samples.

### CROSS REFERENCE TO RELATED PATENT APPLICATIONS

This US Utility Application claims the benefit of, and priority to, U.S. Provisional Patent Application No. 63/769,224 filed on Mar. 10, 2025, the disclosure of which is hereby incorporated by reference in its entirety.

### FIELD

The present technology relates to testing and analyzing water separation characteristics of hydrocarbon fluids and, more particularly, to methods and apparatus for detecting surfactants in fuels by determining the effectiveness of water removal from fuel samples. cl INTRODUCTION

This section provides background information related to the present disclosure which is not necessarily prior art.

Water separation characteristics of hydrocarbon fuels, particularly jet and diesel fuels, can be significantly compromised when surface active agents (surfactants) are introduced into the fuels. These surfactants can infiltrate the fuel during various stages including production, storage, or distribution processes, leading to potential complications in fuel quality and performance.

The detection of surfactants in fuels relies on determining the effectiveness of removing undissolved (free) water from the fuel through filter separator systems. This process involves testing procedures where measured quantities of fuel and distilled water must be precisely emulsified and then passed through specialized equipment.

Testing methods require a series of timed events where specific quantities of fuel and distilled water are carefully emulsified before being passed through a coalescer cell. The effectiveness of water separation is then evaluated using a turbidimeter to obtain a numerical rating (MSEP), where higher ratings indicate better water coalescence and lower surfactant presence in the fuel sample.

Certain apparatus for testing water separation characteristics involve multiple components and procedures. These systems often require significant operator expertise to properly conduct the tests and interpret the results, which can lead to inconsistencies in testing procedures and potential errors in measurement.

Certain testing methods face challenges in providing consistent and reliable results, particularly when dealing with transient hydrocarbon fuels. The accuracy of these tests can be affected by various factors, including the proper formation of water/fuel emulsions and the effectiveness of the coalescing elements used in the testing process.

There is a continuing need for improved methods and apparatus for determining water separation characteristics in hydrocarbon fuels that can provide more reliable and consistent results. Desirably, such improvements would minimize the required operator skill level, reduce the complexity of testing procedures, and provide more accurate measurements of surfactant presence in fuel samples.

### SUMMARY

In concordance with the instant disclosure, improved methods and apparatus for determining water separation characteristics in hydrocarbon fuels that can provide more reliable and consistent results have surprisingly been discovered.

The present technology includes articles of manufacture, systems, and processes that relate to determining water separation characteristics of hydrocarbon fluids through testing methods and apparatus for detecting surfactants by evaluating water coalescence effectiveness in fuel samples.

In one embodiment, a coalescing element for separating water from hydrocarbon fluids includes a first body portion having a fluid conduit with an inlet and associated outlet, a second body portion having a fluid conduit with an inlet and associated outlet, and a filter separator pad. The second body portion is adapted to be interconnected with the first body portion such that the separator pad is disposed between the outlet of the second body portion and the inlet of the first body portion. The first body portion includes a cylindrical wall having an open top and a closed bottom through which the fluid conduit travels, with the second body portion being received within the cylindrical wall of the first body portion. The fluid conduits of both body portions are aligned with each other, and the first and second body portions are interconnected in a fluid-tight relationship using an O-ring seal. The filter separator pad is formed of fiberglass and is disc-shaped, including multiple stacked disc-shaped elements. At least one of the stacked disc-shaped elements is reinforced by a scrim material. Specifically, the coalescer includes two discs.

In another embodiment, the present technology may improve the detection and measurement of surfactants in hydrocarbon fuels by providing simplified methods and apparatus for evaluating water separation characteristics, reducing operator skill requirements, enhancing the reliability of water coalescence testing procedures, and enabling more accurate assessment of water separation effectiveness in transient fuel samples. Water separation characteristics of hydrocarbon fluids such as jet and diesel fuels, for example, may be degraded when surface active agents, referred to herein as surfactants, may be introduced into the fluid during production, storage, or distribution.

The presence of surfactants may typically be detected by determining how effectively undissolved, or free, water may be removed from the hydrocarbon fluid when the hydrocarbon fluid passes through a filter separator. It has been found that by emulsifying measured quantities of fuel and distilled water and passing the emulsion through a coalescer element 100, a numerical rating referred to as MSEP, based on the amount of uncoalesced water remaining in the fuel sample, may be obtained. High minimal ratings may indicate ease of water coalescence, and the fuel may be relatively free of surfactants. The coalescer element, a system incorporating the coalescer element, and a method for determining water separation characteristics, each described in greater detail herein below, together provide a comprehensive and reliable solution for surfactant detection in hydrocarbon fuels.

In one embodiment, a coalescer element for determining water separation characteristics of hydrocarbon fluids may include an outer body having a cylindrical outer wall and a bottom wall defining a hollow interior, with the outer wall including an annular rim extending around an upper perimeter thereof, and the bottom wall having a tubular outlet extending therethrough establishing fluid communication between the hollow interior and an exterior of the outer body. The coalescer element may further include an inner body disposed within the hollow interior of the outer body, the inner body having a hollow interior and a hollow upstanding concentric central member defining a fluid conduit with an inlet and an outlet, with the fluid conduit of the inner body being aligned with the tubular outlet of the outer body. An elastomeric seal may be disposed between the inner body and the outer body to create a fluid-tight seal therebetween, and at least one coalescer disc may be disposed between the inner body and the bottom wall of the outer body, such that a water and fuel emulsion passed through the coalescer element may be directed through the fluid conduit of the inner body, through the at least one coalescer disc, and out through the tubular outlet for collection and subsequent measurement of water separability as an indicator of surfactant presence in the hydrocarbon fuel.

In another embodiment, a system for determining water separation characteristics of hydrocarbon fluids may include the coalescer element in combination with a syringe configured to contain a water and fuel emulsion, wherein the coalescer element may be affixed to an inlet/outlet end of the syringe by fitting the syringe over an upstanding inlet of the fluid conduit of the inner body, and wherein the upstanding inlet may be configured to allow the syringe to slide off of the coalescer element in the event that pressure applied thereto may exceed a predetermined threshold, thereby preventing over-pressurization of the coalescer element during operation of the system. The system may further include a collection container coupled to the tubular outlet of the outer body and configured to receive a treated emulsion discharged from the coalescer element following passage of the water and fuel emulsion through the at least one coalescer disc, with the fluid-tight seal created by the elastomeric seal between the inner body and the outer body ensuring that the water and fuel emulsion may flow properly through the intended path from the fluid conduit of the inner body, through the at least one coalescer disc, and out through the tubular outlet. The system may additionally include a photocell and an associated meter configured to measure the water separability of the treated emulsion collected in the collection container to obtain a numerical water separation rating, wherein higher numerical ratings may indicate better water coalescence and a lower presence of surfactants in the hydrocarbon fuel sample being tested, thereby providing a simplified and reliable testing platform that may minimize required operator skill level and may reduce the complexity of testing procedures.

In yet another embodiment, a method for determining water separation characteristics of hydrocarbon fluids may include a step of injecting a measured quantity of distilled water into a syringe containing a measured quantity of a hydrocarbon fuel to form a water and fuel mixture, followed by a step of mixing the water and fuel mixture for a predetermined time period at a predetermined rate by rapidly agitating the mixture within the syringe to form a water and fuel emulsion suitable for testing, and a step of affixing a coalescer element to an inlet/outlet end of the syringe by fitting the syringe over an upstanding inlet of a fluid conduit of an inner body of the coalescer element. The method may further include a step of passing the water and fuel emulsion from the syringe through the fluid conduit of the inner body and through at least one coalescer disc at a substantially constant rate, wherein a fluid-tight seal created by an elastomeric seal between the inner body and an outer body of the coalescer element may ensure that the water and fuel emulsion flows properly through the intended path, and a step of collecting the treated emulsion discharged through a tubular outlet of the outer body into a collection container for subsequent measurement of water separability. The method may additionally include a step of measuring the water separability rating of the collected treated emulsion using a photocell and associated meter to obtain a numerical water separation rating, wherein a higher numerical rating may indicate greater ease of water coalescence and a lower presence of surfactants in the hydrocarbon fuel sample, and wherein following the measuring step, the coalescer element may be disposed of as a single-use unit to prevent cross-contamination in any subsequent testing.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of the assembled coalescer element with portions cut away to depict internal structure; and

FIG. 2 is an exploded view of the coalescer element of FIG. 1.

FIG. 3 depicts a schematic flow chart illustrating the method steps of the present disclosure.

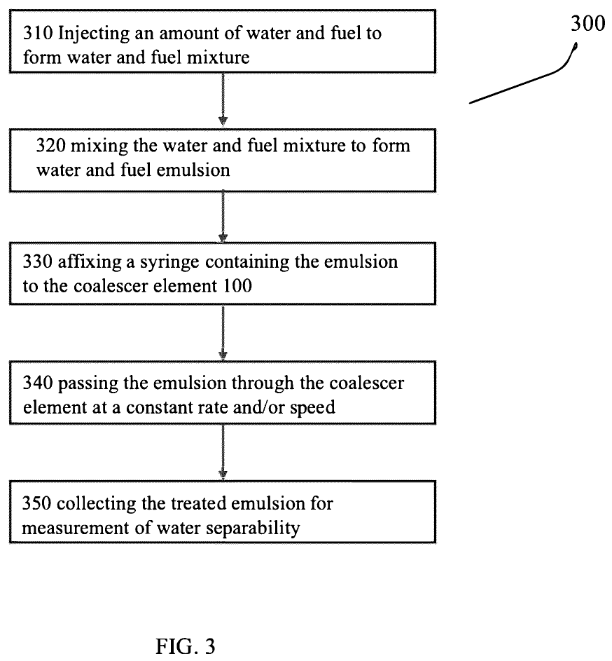

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present technology improves the detection and measurement of surfactants in hydrocarbon fuels by providing simplified methods and apparatus for evaluating water separation characteristics, reducing operator skill requirements, enhancing the reliability of water coalescence testing procedures, and enabling more accurate assessment of water separation effectiveness in transient fuel samples. Water separation characteristics of hydrocarbon fluids such as jet and diesel fuels, for example, can be degraded when surface active agents (surfactants) are introduced into the fluid during production, storage, or distribution. The presence of surfactants may typically be detected by determining how effectively undissolved (free) water can be removed from the hydrocarbon fluid when the hydrocarbon fluid is passed through a filter separator. It has been found that by emulsifying measured quantities of fuel and distilled water and passing the emulsion through a coalescer element, a numerical rating (MSEP) based on the amount of uncoalesced water remaining in the fuel sample, may be obtained. High minimal ratings indicate ease of water coalescence and the fuel is relatively free of surfactants.

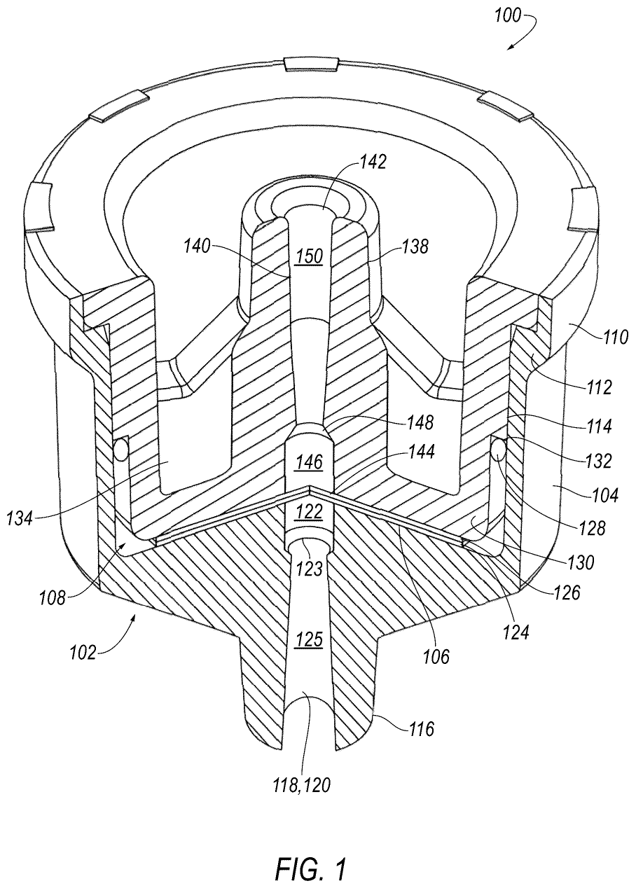

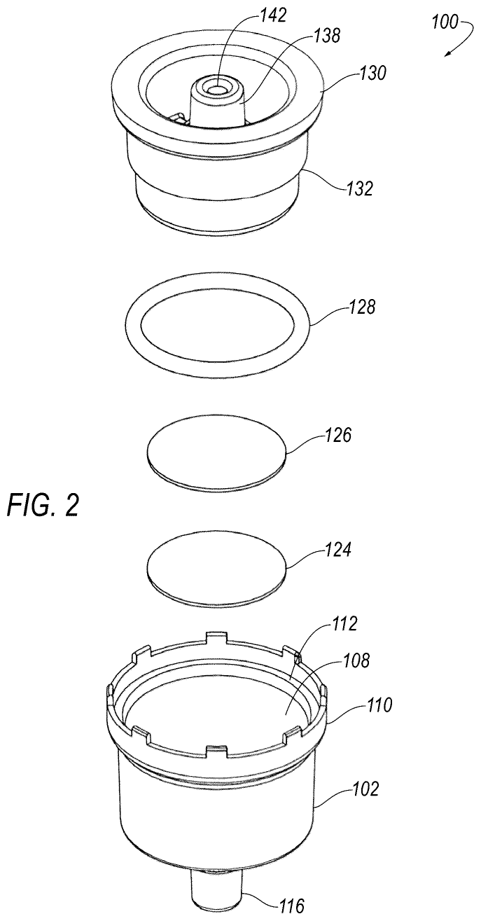

As shown in FIGS. 1-2, a coalescer element 100 is shown. The coalescer element 100 can include an outer body 102 that can be fabricated as a disposable unit for single-use applications. The outer body 102 can be cast from any suitable metal materials, for example, metals resistant to corrosion, metal alloys, for example, aluminum alloy material, which can provide an optimal combination of durability and fluid handling capabilities. Aluminum alloy is particularly desirable for this application due to corrosion resistance when exposed to hydrocarbon fuels, a high strength-to-weight ratio allowing for easy handling during testing procedures, and an ability to be precisely cast to maintain dimensional tolerances required for reliable sealing and fluid flow characteristics.

The outer body 102 can be generally cylindrical in configuration, having an outer wall 104 and a bottom wall 106. The outer wall 104 and bottom wall 106 can define a generally hollow interior 108. The hollow interior 108 can be dimensioned to receive and house internal filtering components of the coalescer element 100 while maintaining proper spacing for fluid flow during testing operations.

The outer wall 104 can include an annular rim 110 that can extend around an upper perimeter of the outer wall 104. The annular rim 110 can define the upper area of the outer wall 104, and can include an interior shoulder 112 formed on an interior surface 114 thereof. The interior shoulder 112 can extend continuously around the interior surface 114 of the outer wall 104 and can be positioned at a predetermined distance from the free edge of the outer wall 104. The interior shoulder 112 can be configured to provide a seating surface for internal components of the coalescer element 100. The interior shoulder 112 can have a predetermined width and depth suitable for supporting and properly positioning components within the hollow interior 108 of the outer body 102. The interior shoulder 112 can be configured to provide a seating surface for internal components of the coalescer element 100. The interior shoulder 112 can have a predetermined width and depth suitable for supporting and properly positioning components within the hollow interior 108 of the outer body 102.

The tubular outlet 116 can include external threading formed on its outer surface to enable secure attachment to a collection container. Additionally, the tubular outlet 116 can include an annular groove formed near its end 126 that is configured to receive a sealing member, such as an O-ring, to create a fluid-tight seal between the tubular outlet 116 and a collection container. The bottom wall 106 can have a generally funnel-shaped configuration that tapers downward toward the tubular outlet 116 to facilitate proper positioning and stable resting of the coalescer element on a collection container during testing procedures. The fluid communication can enable the flow of hydrocarbon fluids through the coalescer element 100 during water separation testing procedures.

In particular, an inner surface 118 of the tubular outlet 116 can define a fluid conduit 120 of the outer body 102. The fluid conduit 120 can include a cylindrical section 122 having a substantially constant cross-sectional diameter along a length. The fluid conduit 120 can include an interior ledge 123 that can extend radially inward from the inner surface 118. It should be understood that the termination of the interior ledge 123 can provide a diameter that is smaller than the substantially constant cross-sectional diameter of the fluid conduit 120. The interior ledge 123 can be positioned at a predetermined distance from the bottom wall 106. A tapered portion 125 can extend from the termination of the interior ledge 123 toward an end of the tubular outlet 116, wherein the cross-sectional diameter of the fluid conduit 120 can generally increase along its length until reaching a maximum or a desired diameter at the end of the tubular outlet 116. The tapered portion 125 can facilitate proper fluid flow characteristics during water separation testing procedures.

A pair of coalescer discs 124, 126 can be disposed in a stack on the bottom wall 106 of the outer body 102, in other words, the coalescer discs 124, 126 can be stacked on top of each other in layers on the bottom wall 106. The coalescer discs 124, 126 can be securely held against the bottom wall 106. The upper coalescer disc 126 can have a thickness of approximately 0.024 inches and a radius of approximately 7/16 inches, as a non-limiting example. The upper coalescer disc 126 can include an upper layer of cured fiberglass and a lower layer of scrim. The lower coalescer disc 124 can have a thickness of approximately 0.017 inches and a radius of approximately 7/16 inches, as a non-limiting example. The disc 124 can include an upper layer of cured fiberglass and a lower layer of scrim. The coalescer discs 124, 126 can be formed of material punched from commercially available products manufactured by Hollingsworth & Vose, specifically from products designated as LD-2021 O-A and LA-8141 O-A, respectively, as non-limiting examples.

An inner body 130 disposed within the outer body 102. The inner body 130 can be generally cylindrical in configuration and can be sized to be received within the hollow interior 108 of the outer body 102. The inner body 130 can be cast from aluminum alloy material to provide optimal structural integrity and fluid handling capabilities. The inner body 130 can include a downwardly facing shoulder 132 that can be adapted to receive an elastomeric O-ring 128. The O-ring 128 can be effective to produce a fluid-tight seal between the interior surface 114 of the hollow interior 108 of the outer body 102 and an outer surface of the inner body 130.

The outer body 102 can include a plurality of crown members 152 that extend upwardly from the annular rim 110. The crown members 152 can be configured to be plastically deformed inwardly toward the hollow interior 108 to mechanically secure the outer body 102 to the inner body 130. When deformed inwardly, the crown members 152 can engage with the upper surface of the inner body 130, creating a mechanical interlock that maintains proper positioning between the outer body 102 and inner body 130. This mechanical staking arrangement, in combination with the O-ring 128 seated in the downwardly facing shoulder 132 of the inner body 130, ensures proper alignment of the fluid conduits and maintains the fluid-tight seal between the inner body 130 and outer body 102 during operation. The sealed interface between the inner body 130 and outer body 102 prevents fluid from bypassing the coalescer discs 124, 126, thereby directing the full flow of the hydrocarbon fluid through the intended flow path from the second fluid conduit 140, through the coalescer discs 124, 126, and into the fluid conduit 120 for collection.

This mechanical staking arrangement, in combination with the O-ring 128 seated in the downwardly facing shoulder 132, ensures proper alignment of the fluid conduits and maintains the fluid-tight seal between the inner body 130 and outer body 102 during operation. The sealed interface between the inner body 130 and outer body 102 prevents fluid from bypassing the coalescer discs 124, 126, thereby directing the full flow of the hydrocarbon fluid through the intended flow path from the second fluid conduit 140, through the coalescer discs 124, 126, and into the fluid conduit 120 for collection.

The inner body 130 can be provided with a hollow interior 134 and a hollow upstanding concentric central member 138. The hollow upstanding concentric central member 138 can be disposed within the hollow interior 134 of the inner body 130. An interior of the hollow upstanding concentric central member 138 can define a second fluid conduit 140. The second fluid conduit 140 can have an inlet 142 at one end and an outlet 144 at an opposite end. In the assembled form, the fluid conduit 140 of the inner body 130 can be aligned with the fluid conduit 120 of the outer body 102 with the coalescer discs 124, 126 disposed therebetween.

The second fluid conduit 140 can include a cylindrical section 146 positioned adjacent to the outlet 144. The cylindrical section 146 can have a substantially constant cross-sectional diameter along its length. It should be understood that the substantially constant cross-sectional diameter of the cylindrical section 146 can be substantially the same diameter as the diameter of the as the cylindrical section 122 of the outer body 102. An interior ledge 148 can extend radially inward from an inner surface of the second fluid conduit 140. It should be understood that the termination of the interior ledge 148 can provide a diameter that is smaller than the substantially constant cross-sectional diameter of the cylindrical section 146. The interior ledge 148 can be positioned at a predetermined distance from the inlet 142. A tapered portion 150 can extend from the termination of the interior ledge 148 toward the inlet 142, wherein the cross-sectional diameter of the second fluid conduit 140 can generally increase along its length until reaching a maximum or a desired diameter at the inlet 142. The tapered portion 150 can facilitate proper fluid flow characteristics during water separation testing procedures.

Referring now to the system 200 of the present technology, the system 200 may include the coalescer element 100 in combination with a syringe configured to contain a water and fuel emulsion, a collection container coupled to the tubular outlet 116 of the outer body 102 for receiving treated emulsion discharged from the coalescer element 100, and a photocell and associated meter configured to measure the water separability of the treated emulsion to obtain a numerical water separation rating. The inlet 142 of the second fluid conduit 140 may be configured as an upstanding inlet such that the syringe may be fitted over the upstanding inlet 142 and may also slide off of the coalescer element 100 in the event that pressure applied to the coalescer element 100 may exceed a predetermined threshold, thereby preventing over-pressurization of the coalescer element 100 during operation. The components of the system 200 may work together to provide a simplified, reliable, and repeatable testing platform that may minimize required operator skill level and reduce the complexity of testing procedures associated with prior art approaches. The photocell and associated meter of the system 200 may obtain the numerical MSEP rating, wherein higher ratings may indicate better water coalescence and a lower presence of surfactants in the fuel sample being tested.

Referring now to the method 300 of the present technology, the method 300 may provide a reliable and consistent procedure for determining water separation characteristics of hydrocarbon fluids using the coalescer element 100 and the system 200 described herein.

In operation, a measured quantity of water can be injected into a syringe (not shown) filled with a given quantity of fuel to be tested. The water and fuel sample can be rapidly mixed for a predetermined time period to form a water/fuel emulsion. The coalescer element 100 can then be affixed to the inlet/outlet end of the syringe. The syringe inlet/outlet can be fitted over the upstanding inlet 142 of the fluid conduit 140 of the inner body 130. Importantly, the upstanding inlet is configured such that the syringe can slide off of the coalescer element 100 in the event that pressure on the element 100 is too high. Advantageously, this configuration can militate against the element 100 from being over pressurized, in operation.

The water/fuel emulsion can be forced from the syringe through the fluid conduit 140 of the inner body 130. The emulsion can then pass through the upper and lower coalescer discs 124, 126 which are held in a stacked arrangement between the inner body 130 and outer body 102. After passage through the coalescer discs 124, 126, the treated emulsion can be ejected through the fluid conduit 120 and tubular outlet 116 of the outer body 102. The treated emulsion can then be collected in a test container.

The water separability of the collected sample fuel can be measured using a photocell and an associated meter to obtain a numerical rating (MSEP). Higher ratings can indicate better water coalescence and lower presence of surfactants in the fuel sample. The fluid-tight seal created by the O-ring 128 between the inner body 130 and outer body 102 can ensure that the water/fuel emulsion flows properly through the intended path from the inlet 142, through the coalescer discs 124, 126, and out through the tubular outlet 116 during the testing operation.

In some embodiments, the method 300 of the present disclosure provides a step 310 of injecting a measured quantity of distilled water into a syringe containing a measured quantity of a hydrocarbon fuel to form a water and fuel mixture. The method 300 may further include a step 320 of mixing the water and fuel mixture for a predetermined time period at a predetermined rate, for example, at a predetermined rate between 10,000 rpm and 50,000 rpm, and all values therebetween, or at a predetermined rate such that it is at least fast enough that any preexisting coagulants, or comingled surfactant/water/fuel emulsions are sheared apart and the subsequent sample ends up having a more uniform consistency when it is pushed through the coalescing cell, such that the predetermined rate provides that the water/fuel mixture is rapidly agitated within the syringe, to form a water and fuel emulsion suitable for testing. Following the step 320, the method 300 may include a step 330 of affixing the coalescer element 100 to an inlet/outlet end of the syringe by fitting the syringe over the upstanding inlet 142 of the second fluid conduit 140 of the inner body 130. The method 300 may then include a step 340 of passing the water and fuel emulsion from the syringe through the second fluid conduit 140 of the inner body 130 and through the coalescer discs 124 and 126 at a substantially constant rate, and/or speed, wherein the fluid-tight seal created by the O-ring 128 between the inner body 130 and outer body 102 may ensure that the water and fuel emulsion flows properly through the intended path. After completion of the step 340, the method 300 may include a step 350 of collecting the treated emulsion discharged through the fluid conduit 120 and tubular outlet 116 of the outer body 102 into a collection container for subsequent measurement. The method 300 may further include a step 360 of measuring the water separability rating of the collected treated emulsion using a photocell and associated meter to obtain a numerical MSEP rating, wherein a higher numerical rating may indicate greater ease of water coalescence and a lower presence of surfactants in the hydrocarbon fuel sample, and wherein the coalescer element 100 may thereafter be disposed of as a single-use unit to prevent cross-contamination in subsequent testing.

In some embodiments, the method 300 of the present disclosure provides a method for determining water separation characteristics of hydrocarbon fluids. In various embodiments, the method comprises:
(a) injecting a measured quantity of water into a syringe containing a quantity of fuel to form a water/fuel mixture;
(b) mixing the water/fuel mixture for a predetermined time period at a predetermined rate to form an emulsion;
(c) affixing a coalescer element to an inlet/outlet end of the syringe, the coalescer element including an outer body having a hollow interior and a tubular outlet, an inner body disposed within the hollow interior of the outer body, and at least one coalescer disc disposed between the inner body and outer body;
(d) passing the emulsion through the coalescer element at a constant rate and/or speed; and
(e) collecting the treated emulsion for measurement of water separability.

Example embodiments may be provided so that this disclosure may be thorough and may fully convey the scope to those who may be skilled in the art. Numerous specific details may be set forth such as examples of specific components, devices, and methods, to provide a thorough understanding of embodiments of the present disclosure. It may be apparent to those skilled in the art that specific details need not be employed, that example embodiments may be embodied in many different forms, and that neither should be construed to limit the scope of the disclosure. In some example embodiments, well-known processes, well-known device structures, and well-known technologies may not be described in detail. Equivalent changes, modifications and variations of some embodiments, materials, compositions and methods may be made within the scope of the present technology, with substantially similar results.

### EXAMPLES

The following examples demonstrate various embodiments and implementations of a coalescer element for separating water from hydrocarbon fuels. These examples illustrate specific configurations and testing procedures that highlight the effectiveness of the coalescer element design features in detecting and measuring surfactants in hydrocarbon fuels. The examples particularly demonstrate how the structural components work together to enable reliable water separation testing, including the interaction between the outer and inner bodies, the specialized coalescer discs, and the fluid flow characteristics through the aligned conduits. Each example focuses on different aspects of the technology while maintaining the fundamental goal of providing simplified methods for evaluating water separation characteristics and enhancing the reliability of water coalescence testing procedures.

### Example 1: Basic Water Separation Testing

In this example, a coalescer element 100 was assembled using an aluminum alloy outer body 102 with a cylindrical configuration having an outer wall 104 and bottom wall 106 defining a hollow interior 108. The outer body 102 included an annular rim 110 around the upper perimeter with an interior shoulder 112 formed on the interior surface 114.

Two coalescer discs 124, 126 were installed in a stack on the bottom wall 106. The upper disc 124 was formed from LD-2021 O-A material with a thickness of 0.024 inches and a radius of 7/16 inches, including an upper layer of cured fiberglass and a lower layer of scrim. The lower disc 126 was formed from LA-8141 O-A material with a thickness of 0.017 inches and the same radius, also featuring an upper fiberglass layer and lower scrim layer.

A measured quantity of distilled water was injected into a syringe containing the test fuel sample. The mixture was rapidly agitated to create a water/fuel emulsion. The coalescer element 100 was then attached to the syringe, and, utilizing the syringe the emulsion was then forced to flow through the coalescer element 100 to treat test fuel sample by passing it through the two coalescer discs 124, 126. The treated sample was collected in a collection container is it flowed out of the tubular outlet 116 of the outer body 102. The treaded sample was analyzed using a photocell and meter to determine the water separability rating.

### Example 2: Fluid-Tight Seal Configuration

In this example, the coalescer element 100 was assembled with particular attention to the sealing mechanism. The inner body 130 was cast from aluminum alloy and included a downwardly facing shoulder 132 specifically adapted to receive an elastomeric O-ring 128. The inner body 130 was carefully positioned within the outer body's hollow interior 108.

The O-ring 128 was installed on the downwardly facing shoulder 132 of the inner body 130, creating a fluid-tight seal between the interior surface 114 of the hollow interior 108 of the outer body 102 and the outer surface of the inner body 130. The inner body 130 was then secured to the outer body 102 through the described staking process to maintain proper alignment and seal integrity.

The assembled unit demonstrated effective fluid handling through aligned fluid conduits 118, 140. The inner body's fluid conduit 140 included a cylindrical section 146 with a constant cross-sectional diameter, an interior ledge 148 extending radially inward, and a tapered portion 150 extending toward the outlet 144, facilitating proper fluid flow characteristics during testing.

### Example 3: Advanced Flow Configuration

In this example, the coalescer element 100 was configured with specific attention to flow characteristics. The tubular outlet 116 extending from the bottom wall 106 was positioned centrally and included a fluid conduit 120 with three distinct sections: a cylindrical section 122 with constant cross-sectional diameter, an interior ledge 123 at a predetermined distance from the outlet and extending radially inward, and a tapered portion 125 extending from the interion ledge 123 toward the end 126.

The inner body 130 was installed with its hollow upstanding concentric central member 138 precisely aligned with the outer body's fluid conduit 120. The second fluid conduit 140 featured a similar three-section configuration: cylindrical section 146 adjacent to the inlet 142, an interior ledge 148 at a predetermined distance from the inlet and extending radially inward, and tapered portion 150 extending from the interion ledge 148 toward the outlet 144.

When tested with a water/fuel emulsion, this configuration demonstrated effective water separation capabilities. The treated emulsion passed sequentially through the aligned fluid conduits and coalescer discs 124, 126 before being ejected through the tubular outlet 116. The collected sample showed measurable water separation characteristics when evaluated using standard testing equipment.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A coalescer element for determining water separation characteristics comprising:
an outer body having an outer wall and a bottom wall defining a hollow interior;
a tubular outlet extending through the bottom wall to establish fluid communication between the hollow interior and an exterior of the outer body; an annular rim extending around an upper perimeter of the outer wall;
an inner body disposed within the hollow interior of the outer body and having a fluid conduit aligned with the tubular outlet of the outer body, the inner body having:
a hollow interior; a hollow upstanding concentric central member disposed within the hollow interior of the inner body and defining the fluid conduit with an inlet and an outlet; a downwardly facing shoulder adapted to receive an elastomeric seal; and
at least one coalescer disc disposed in a stack between the inner body and the bottom wall of the outer body.

2. The coalescer element of claim 1, wherein the outer body is cast from an aluminum alloy material.

3. The coalescer element of claim 1, wherein the inner body includes a downwardly facing shoulder adapted to receive the elastomeric seal, and the elastomeric seal is an O-ring.

4. The coalescer element of claim 1, wherein the annular rim includes a plurality of crown members extending upwardly therefrom, the crown members being plastically deformed inwardly to mechanically secure the inner body to the outer body.

5. The coalescer element of claim 1, wherein the at least one coalescer disc comprises two coalescer discs stacked on top of one another on the bottom wall of the outer body, each of the two coalescer discs being disc-shaped and formed of fiberglass, and at least one of the two coalescer discs including a scrim reinforcement layer.

6. The coalescer element of claim 5, wherein a first coalescer disc of the two coalescer discs has a thickness of approximately 0.024 inches and a second coalescer disc of the two coalescer discs has a thickness of approximately 0.017 inches, each coalescer disc having a radius of approximately 7/16 inches.

7. The coalescer element of claim 1, wherein the tubular outlet includes external threading on an outer surface thereof and an annular groove configured to receive a sealing member to create a fluid-tight seal between the tubular outlet and a collection container, and wherein the bottom wall has a funnel-shaped configuration that tapers downward toward the tubular outlet.

8. A system for determining water separation characteristics of hydrocarbon fluids, comprising:
a syringe configured to contain a water and fuel emulsion; and
a coalescer element affixed to an inlet/outlet end of the syringe, the coalescer element comprising:
an outer body having a cylindrical outer wall and a bottom wall defining a hollow interior, the bottom wall having a tubular outlet extending therethrough establishing fluid communication between the hollow interior and an exterior of the outer body;
an inner body disposed within the hollow interior of the outer body, the inner body having a fluid conduit with an inlet and an outlet, the fluid conduit of the inner body being aligned with the tubular outlet of the outer body, the inlet of the fluid conduit being configured to receive the inlet/outlet end of the syringe;
an elastomeric seal disposed between the inner body and the outer body creating a fluid-tight seal therebetween; and
at least one coalescer disc disposed between the inner body and the bottom wall of the outer body, wherein the emulsion passes from the syringe through the fluid conduit of the inner body, through the at least one coalescer disc, and out through the tubular outlet of the outer body.

9. The system of claim 8, further comprising a collection container coupled to the tubular outlet of the outer body and configured to receive a treated emulsion discharged from the coalescer element.

10. The system of claim 9, further comprising a photocell and an associated meter configured to measure water separability of the treated emulsion collected in the collection container to obtain a numerical water separation rating.

11. The system of claim 8, wherein the inlet of the fluid conduit of the inner body is an upstanding inlet configured to allow the syringe to slide off of the coalescer element when pressure on the coalescer element exceeds a predetermined threshold, thereby preventing over-pressurization of the coalescer element.

12. The system of claim 8, wherein the at least one coalescer disc comprises two coalescer discs stacked on top of one another on the bottom wall of the outer body, each coalescer disc being formed of fiberglass and including a scrim reinforcement layer.

13. The system of claim 8, wherein the inner body is secured to the outer body by a plurality of crown members extending upwardly from annular rim of the outer wall, the crown members being plastically deformed inwardly to mechanically engage an upper surface of the inner body.

14. The system of claim 8, wherein the outer body and the inner body are each cast from an aluminum alloy material, and wherein the fluid conduit of the inner body includes a tapered portion extending toward the inlet to facilitate fluid flow of the emulsion from the syringe into the coalescer element.

15. A method for determining water separation characteristics of hydrocarbon fluids comprising:
injecting a measured quantity of water into a syringe containing a quantity of fuel to form a water/fuel mixture;
mixing the water/fuel mixture for a predetermined time period at a predetermined rate to form an emulsion;
affixing a coalescer element to an inlet/outlet end of the syringe, the coalescer element including an outer body having a hollow interior and a tubular outlet, an inner body disposed within the hollow interior of the outer body, and at least one coalescer disc disposed between the inner body and outer body;
passing the emulsion through the coalescer element at a constant rate and/or speed; and
collecting the treated emulsion for measurement of water separability.

16. The method of claim 15, further comprising measuring a water separability rating of the collected treated emulsion using a photocell and an associated meter to obtain a numerical rating indicative of a level of surfactant presence in the hydrocarbon fuel.

17. The method of claim 16, wherein a higher numerical rating indicates greater ease of water coalescence and a lower presence of surfactants in the hydrocarbon fuel sample.

18. The method of claim 15, wherein the mixing step comprises rapidly agitating the water and fuel mixture in the syringe to produce the water and fuel emulsion prior to affixing the coalescer element to the syringe.

19. The method of claim 15, wherein the at least one coalescer disc comprises two coalescer discs stacked on the bottom wall of the outer body, each coalescer disc being formed of fiberglass, and the emulsion passes sequentially through the two coalescer discs during the passing step.

20. The method of claim 15, wherein the coalescer element is configured as a single-use disposable unit, and the method further comprises disposing of the coalescer element following collection of the treated emulsion.
